(19) United States
(12) Patent Application Publication
(10) Pub. No.: US 2023/0121385 A1
(43) Pub. Date: Apr. 20, 2023

(54) IMPROVING SPECIFICITY OF NON-PHYSIOLOGICAL SHORT INTERVALS AS A LEAD MONITORING DIAGNOSTIC

(71) Applicant: Lambda Nu Technologies LLC, Orono, MN (US)

(72) Inventor: Charles Dennis Swerdlow, Los Angeles, CA (US)

(21) Appl. No.: 17/898,226

(22) Filed: Aug. 29, 2022

Related U.S. Application Data
(60) Provisional application No. 63/238,262, filed on Aug. 30, 2021.

(51) Int. Cl. G01R 27/16; A61N 1/362; A61N 1/372

(52) U.S. Cl. CPC G01R 27/16; A61N 1/362; A61N 1/372

(57) ABSTRACT

Methods and systems for diagnosis of lead system anomalies for an implantable medical device. More particularly, the present disclosure relates to prediction and/or detection of a lead system condition by utilizing electrogram (EGM) analysis to identify which non-physiological short interval signals (NPSIs) are more indicative of lead system conditions, including lead failure, than of other causes.

### RELATED APPLICATION

This application claims the benefit of U.S. Provisional Application No. 63/238,262 filed Aug. 30, 2021, which is hereby fully incorporated herein by reference.

### TECHNICAL FIELD

The present disclosure relates, generally, to methods and systems for diagnosis of lead system anomalies for an implantable medical device. More particularly, the present disclosure relates to prediction and/or detection of a lead system condition by utilizing electrogram (EGM) analysis to identify which non-physiological short interval signals are more indicative of lead system conditions, including lead failure, than of other causes.

### BACKGROUND

The terms lead system condition, lead failure and lead condition refer to conditions in which the implantable lead of a cardiac implantable electronic device (CIED), such as a pacemaker or an implantable cardioverter defibrillator (ICD), is not functioning properly due to structural lead failure and/or connection problem between the lead and the device. Lead system conditions can result in failure to pace, delivery of inappropriate ICD therapy or failure to deliver appropriate ICD therapy, and physical failure of the lead may damage the heart or result in other unpredictable life-threatening events.

The most common causes of defibrillation lead failure involve pace-sense components of the implantable lead that can cause inappropriate shocks due to oversensing or failure of pacing therapy if the lead failure is not identified early. The ideal diagnostic for pace-sense lead failure would have both high sensitivity for early-stage lead failures to provide maximum warning and high specificity to reduce the need for unnecessary rapid responses by the health care system.

Most present diagnostics that monitor for oversensing provide for high sensitivity because sensing is continuously monitored, and oversensing often occurs early during either insulation breach or conductor fracture. Unfortunately, these diagnostics have low specificity and low positive predictive value that can result in an increased number of “false positive” alerts. One approach to addressing these issues is a differential method of EGM analysis as disclosed by the inventor in U.S. Pat. No. 9,486,624. While broader in scope in its applicability to resolving issues with respect to low specificity and low positive predictive value for lead monitoring diagnostics, there can be implementation issues to such a differential method of EGM analysis that may limit the adoption of this approach.

Another approach to addressing these issues involves analysis of isolated fracture-induced signals that have long been known to be a sensitive indicator of early-stage of conductor failure due to conductor fracture or insulation breach which are the most common failure modes of CIED leads. The interval beginning or ending with a fracture-induced signal, for example, may be shorter than the interval between any two successive cardiac sensed events (commonly referred to as device-detected “R waves”), which is constrained by the duration of a physiologic cardiac refractory period. Thus, very-short, sensed intervals near the ventricular blanking period of the CIED do not represent successive cardiac activations, except during ventricular fibrillation. Such intervals are referred to as “non-physiologic” short intervals (NPSIs) because the interval between non-physiologic signals is not constrained by biological refractory periods.

Although single NPSIs are highly sensitive for conductor fracture, their specificity and positive predictive values are too low to make them a useful diagnostic. In fact, the most common cause of “NPSIs” are intervals between two physiologic signals that are not constrained by physiologic refractory periods (e.g. T-P intervals, P-R intervals, or two components of a single, long ventricular EGM and electromagnetic interference (EMI)). See Gunderson BD, et al., “Causes of ventricular oversensing in implantable cardioverter-defibrillators: implications for diagnosis of lead fracture.” Heart Rhythm 2010 May; 7:626-633; Ng J, et al, “Incidence of nonphysiologic short VV intervals detected by the sensing integrity counter with integrated bipolar compared with true bipolar leads,” J Intery Card Electrophysiol, 2014 April; 39(3):281-5; Swerdlow CD, et al, “Downloadable algorithm to reduce inappropriate shocks caused by fractures of implantable cardioverter-defibrillator leads,” Circulation November. 18 2008; 118:2122-2129; Swerdlow C D, et al, “Downloadable software algorithm reduces inappropriate shocks caused by implantable cardioverter-defibrillator lead fractures: a prospective study,” Circulation Oct. 12 2010; 122:1449-1455; Swerdlow CD, et al, “Troubleshooting Implantable Cardioverter Defibrilator Sensing Problems,” Circ Arrhythmia Electrophysiol 2014 December; 114:1237-1261; Ellenbogen K A, et al, “Performance of Lead Integrity Alert to assist in the clinical diagnosis of implantable cardioverter defibrillator lead failures,” Circulation Arrhythmia and Electrophysiology, 2013 December; 6:1169-1177; and Swerdlow CD, et. al, “Preventing overdiagnosis of implantable cardioverter-defibrillator lead fractures using device diagnostics,” J Am Coll Cardiol Jun. 7 2011; 57:2330-A rapidly increasing count of NPSIs is a sensitive but nonspecific indicator of a lead system condition. Present CIEDs use a rapidly increasing count of NPSIs as a diagnostic for lead failures and lead system conditions as described, for example, in U.S. Pat. Nos. 7,369,893 and 9,037,240. For example, in one current CIED a lead system diagnostic uses a count of 30 NPSIs in 3 days. Another current CIED uses a count of 30 NPSIs in 24 hours. Such counts are referred to as a Sensing Integrity Counter (SIC).

Unfortunately, even the SIC criterion of 30 NPSIs in a 72-hour monitoring period has proved too non-specific to be used as a lead failure diagnostic in isolation. To increase specificity, CIED makers utilize the SIC criterion only if an additional oversensing criterion is fulfilled. Yet even with a rapidly increasing count of NPSIs and an additional oversensing criterion, this diagnostic technique still has a relatively low positive predictive value of about 35% (see articles to be published attached as Exhibit A, the disclosure of which is hereby incorporated by reference). Such low positive prediction values reduce the usefulness of this kind of analysis to provide an early indication of lead system failure.

Although some alerts generated by existing NPSI approaches can be triggered by causes other than lead failure that can warn of clinically useful findings such as T-wave oversensing, it would be desirable to provide urgent lead alerts only for lead failures and to provide one or more other categories of alerts for other causes of oversensing. Thus, there is an unmet need for an improved diagnostic for lead system failure that enhances specificity while retaining high sensitivity.

### SUMMARY

In various embodiments, automated analysis of stored intervals and electrogram signals associated with non-physiological short intervals (NPSIs) is utilized to identify those NPSIs more likely to be caused by lead system conditions than other causes of oversensing. NPSIs likely to be caused by conditions other than lead failure are rejected, while those that satisfy additional criteria indicative of lead system conditions are prioritized in relation to their specificity for lead system conditions. In various embodiments, the analysis and criteria form an incremental, hierarchy, identifying NPSIs that are progressively more specific for a lead system condition, and hence provide a progressively more specific CIED diagnostic.

In embodiments, an automated method of identifying a lead condition of an implantable lead system operably positioned to obtain electrical signals from one or more chambers of the heart of a patient and operably coupled to an implanted medical device that includes an automated diagnostic monitoring process implemented by a cardiac implantable electrical device alone or in conjunction with a programmer and/or remote monitoring network. The automated method includes sensing an electrogram signal from an implantable lead and evaluating the electrogram signal for a series of sensed events. For each interval between successive sensed events, the automated method determines whether that interval is a NPSI. A processor system is then used to analyze if the NPSI is a More Specific NPSI for a lead system condition. This analysis is based on whether a measure of the frequency content of the electrogram signals in two analysis windows corresponding to the sensed events that begin and end the NPSI. Once the automated method identifies a predetermined number of More Specific NPSIs within a predetermined monitoring period, a lead system condition alert is generated by the CIED alone or in conjunction with a programmer and/or remote monitoring network.

The analysis of NPSIs in accordance with various embodiments provides an early and sensitive indicator of lead system conditions with higher specificity and positive predictive values than current approaches.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 depicts a multilumen transvenous lead for a cardiac implantable electrical device (CIED) as an example of an implantable lead in accordance with various embodiments.

FIG. 2 illustrates a pulse generator of a CIED connected to a patient's heart via a transvenous lead as an example of an implantable medical device in accordance with various embodiments.

FIG. 3 is an embodiment of a control circuitry and microprocessor(s) within a CIED as an example of an implantable medical device in accordance with various embodiments.

FIG. 4 depicts a simplistic flowchart diagram of an automated method in which a count of More Specific (MS) NPSIs triggers an alert for a lead system condition.

FIG. 5 depicts a flowchart diagram of an automated method that prevents alerts of More Specific NPSIs that are not likely to be caused by a lead system condition.

FIG. 6 depicts a flowchart diagram of an automated method that accelerates alerts for lead system conditions in the presence of additional features that increase specificity of one or a few More Specific NPSIs.

FIG. 7 depicts a flowchart diagram of an automated method that adds an observation or low intensity alert for oversensing that does not have a high likelihood of indicating a lead system condition.

FIG. 8 depicts a flowchart diagram of an automated method that further refines classification of NPSIs that have a low likelihood of indicating a lead system condition.

FIG. 9 depicts a schematic flowchart for an automated method that applies a differential technique to a sequence of baseline rhythm EGMs and stores the resultant output.

FIG. 10 depicts a schematic flowchart for an automated method that utilizes a differential technique for analysis of More Specific NPSIs based on a ratio of the amplitude of the EGM associated with the NPSI after processing by the differential technique to the amplitude of the stored baseline-rhythm EGM after processing by the differential technique.

FIG. 11 illustrates an embodiment of clinical EGM data for a conductor fracture.

FIG. 12 illustrates an embodiment of clinical EGM data for a conductor fracture.

FIG. 13 illustrates an embodiment of clinical EGM data for NPSIs caused by T-wave oversensing.

FIG. 14 illustrates an embodiment of clinical EGM data for NPSIs caused by R-wave double-counting.

FIG. 15 illustrates an embodiment of clinical EGM data for NPSIs caused by EGM signals in ventricular fibrillation.

FIG. 16 illustrates an embodiment of clinical EGM data for NPSIs caused by electromagnetic interference.

FIG. 17 illustrates an embodiment of clinical EGM data for NPSIs caused by oversensing of diaphragmatic myopotentials.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE SPECIFICATION

In various embodiments, a method and system of identifying a lead system condition of an implanted lead operably coupled to an implanted medical device (CEID) that senses electrical activation in one or more chambers of the heart of a patient. The term “lead failure” is understood to encompass lead system conditions that result in oversensing indistinguishable from lead failure, most commonly connection issues between the lead and the implantable medical device header. Such connection problems, especially incomplete insertion of a lead pin into the header, are more common with an older IS-1 leads than newer DF4 leads. In various embodiments, the implantable lead in a lead system may comprise one or more of an intravascular lead, and epicardial lead, a substernal lead, and/or a subcutaneous lead. Thus, in various embodiments the detection of implantable lead failures by utilizing the detection of NPSIs is used in both specific and diffuse manners to gain a more holistic diagnostic picture of the lead system.

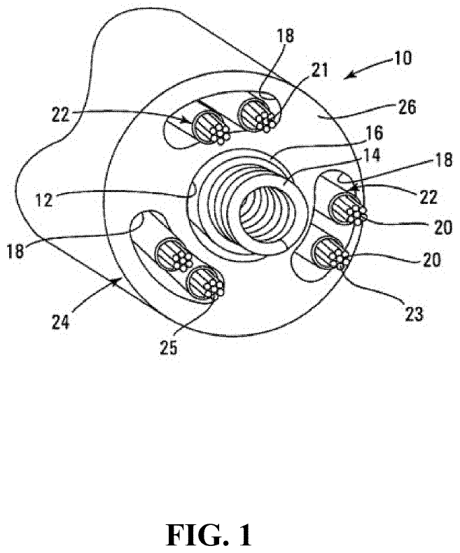

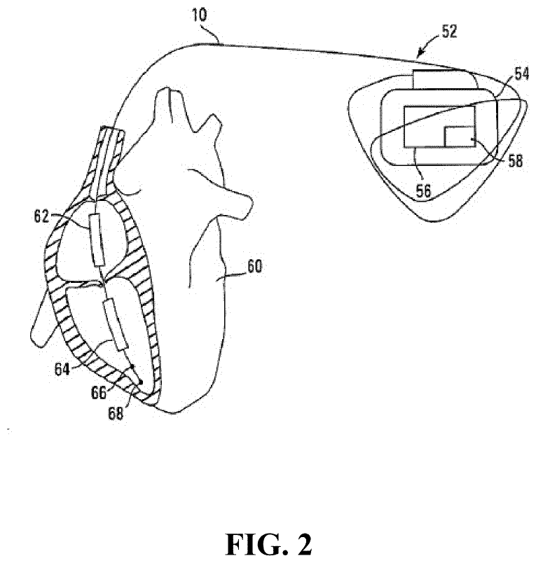

FIG. 1 illustrates one example of an implantable cardiac lead 10. The lead 10 is comprised of a lumen 12 and center inner pacing coil 14 surrounded by polytetrafluoroethylene insulation 16, a plurality of lumens 18 each containing at least one conductor 20 with each conductor 20 surrounded by ethyltetrafluoroetheylene insulation 22, the primary silicone elastomer insulation of the lead body 26 and an optional outer insulating layer 24 usually comprised of polyurethane or a copolymer of silicone and polyurethane. The conductors 20 include a sense conductor 21, a high voltage RV conductor 23 and a high voltage SVC conductor 25. The plurality of lumens 18 is disposed in the silicone insulation 26. The conductors 20 carry biological signals from cardiac electrical signals to the generator and current from the generator to the pace-sense electrodes 66, 68, high voltage RV coil 64 and high voltage SVC coil 62 (FIG. 2).

FIG. 2 depicts an ICD system implanted in the chest of a patient having an outer housing 54, commonly referred to as a “CAN,” inner circuitry 56 and a battery 58. Connection is made to the heart 60 via the lead 10. The lead 10 is often wrapped around the CAN 54 in the pocket until it exits, shown as reference number 52, the pocket on its intravascular course. The lead 10 can have an optional proximal defibrillation coil 62, which is commonly referred to as the SVC Coil 62. The lead 10 also has a distal defibrillation coil 64 or RV Coil 64. Also shown is the optional “ring” pacing-sensing electrode 66. Located at the distal end of the lead 10 is the “tip” pacing-sensing electrode 68.

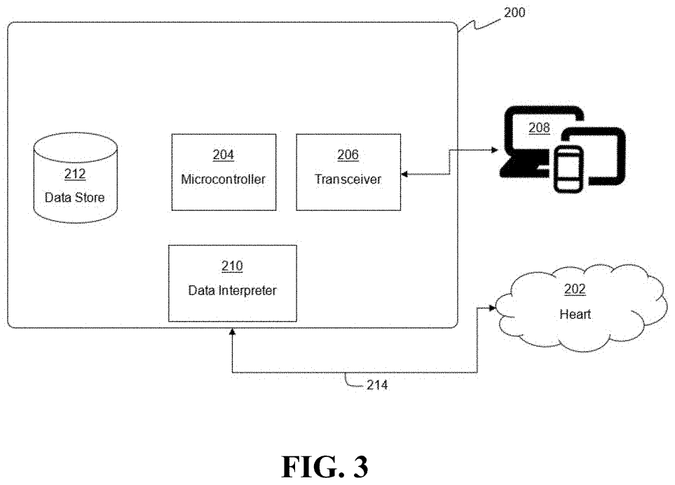

FIG. 3 depicts an embodiment of the control circuitry and microprocessors(s) of an implantable cardioverter defibrillator (ICD) system that is a CIED implanted in the chest of a patient. A heart (202) is made to be operably coupled to the computer architecture of the ICD (200) via a heart lead 214. The heart lead takes in data from the heart and passes it to a data interpreter 210. The data interpreter passes the data to a non-transitory storage medium of a data store 212. The data store passes the non-transitory digital information to a microcontroller 204. The microcontroller detects a fault condition and passes the non-transitory digital information to a transceiver 206. The transceiver transmits the non-transitory digital information about the heart to a physician's device 208. The physician's device can take any number of forms from a remote programmer, to a computer display, or a mobile device.

For further description of various implantable leads and CIEDs such as pacemakers and ICDs, reference is made to the patents and references set forth in the Background Section, the disclosure of each of which is hereby incorporated by reference herein.

Definition of and Classification of Non-physiologic Short Intervals (NPSI)

A Non-Physiologic Short Interval (NPSI) is an Interval Between a First Sensed Event and a second sensed event that is too short to represent successive cardiac depolarizations, except in ventricular fibrillation. Thus, a NPSI indicates oversensing, except during ventricular fibrillation. Operationally, NPSI may be defined based either on the interval between two successive sensed events or on the interval between the end of the blanking period after the first sensed event and the subsequent sensed event. Typical ranges for definitions of NPSIs include intervals between sensed events of less than 140-160 ms or intervals in a range from 20-40 ms after a blanking period following the first sensed event. A NPSI may be expressed as a device-detected interval between two sensed ventricular events (V-V interval), where it is understood that, in general, that at least one event does not represent ventricular activation. Rather, at least one event represents oversensing of physiologic signals (e.g. P waves, T waves, R-wave double-counting, non-cardiac myopotentials) or non-physiologic signals (e.g. EMI or lead-failure related signals).

A temporal analysis window of the continuously-monitored electrogram signal is defined as a time period that uses a sensed event as a fiduciary timing point. As an example, the analysis window may extend from approximately−20 ms before the sensed event to +100 ms after the sensed event. In various embodiments, the electrogram signal in one or more analysis windows is processed to obtain a measure or indication of a frequency content of the electrogram signal around a sensed event. It is understood that the event contains timing information, and the signal contains amplitude, frequency, and other signal-encoded information. It is also understood that processing the electrogram signal corresponding to a sensed event refers to processing the signal in the sensed event's analysis window. It is further understood that this processing is performed after the signal is sampled digitally (e.g., at 256, 512, or 1024 Hz). Thus, processing involves a mathematical operation performed on a sequence of samples corresponding to the analysis window.

A High-Frequency Electrogram Signal (high-frequency EGM) is a signal in an analysis window having a measure of a frequency content that exceeds a first predetermined threshold. In various embodiments, this threshold may be defined in relation to the higher end of the frequency content of the conducted baseline rhythm, corresponding to a threshold in the range of about 40 Hz.

A Low-Frequency Electrogram Signal (low-frequency EGM) is a signal in an analysis window having a measure of a frequency content that is less than a second predetermined threshold. In various embodiments, this threshold is defined in relation to the lower end of the frequency content of the conducted baseline rhythm, corresponding to a threshold in the range of about 15-20 Hz.

A More Specific NPSI is a NPSI that is more likely to indicate a lead system condition than a non-lead failure oversensing event. In various embodiments, a More Specific NPSI is determined based on analysis of the electrogram signals corresponding to the two sensed events that begin and end the NPSI (the “bounding electrogram signals”). In a simpler embodiment, a More Specific NPSI is identified based on a measure of the frequency content of these bounding electrogram signals. In other embodiments, supplementary criteria are applied to further enhance the specificity of More Specific NPSIs.

Determine if a NPSI is a More Specific NPSI.

Electrograms caused by oversensing of cardiac signals (e.g., ventricular fibrillation, P waves, T waves) almost always have lower frequency content than the baseline rhythm. Thus, when oversensing of cardiac signals causes a NPSI, neither electrogram signal in either of the NPSI's analysis windows is a high-frequency EGM, and one of the two electrogram signals is often a low-frequency EGM. In contrast, oversensing caused by lead system conditions typically causes high-frequency EGMs. So, when oversensing from lead system conditions causes a NPSI, the electrogram signal in at least one of the NPSI's two analysis windows usually is a high-frequency EGM, and neither EGM is a low-frequency EGM.

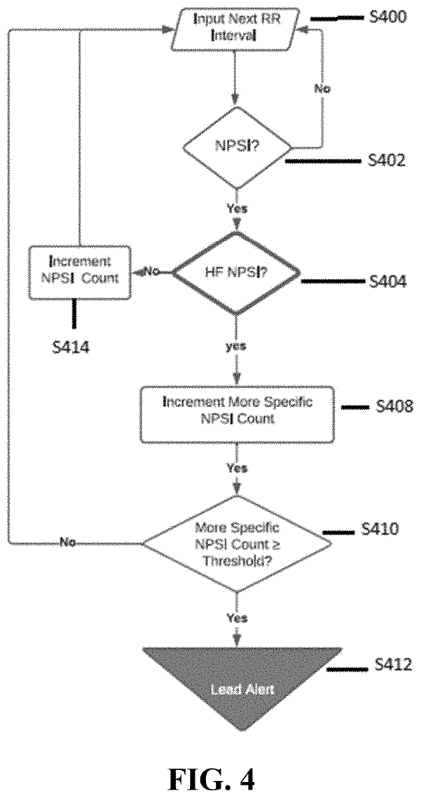

In embodiments, FIG. 4 depicts a simplified embodiment applying these differences in frequency content of oversensed signals to identify More Specific NPSIs. For each NPSI, the frequency content of the electrogram signals in the beginning and ending analysis windows is analyzed, and a measure of this frequency content is determined. An NPSI may be considered a More Specific NPSI if it fulfills either of two criteria: (1) the electrogram signal in at least one of the two analysis windows beginning and ending the interval contains a high-frequency EGM; or (2) the electrogram signals in neither of the two analysis windows beginning and ending the interval contain a low-frequency EGM.

Methods are described herein for determining if an electrogram signal is high-frequency EGM. Similar methods can be used to determine if an electrogram signal is a low-frequency EGM. A high-frequency EGM may be determined by a direct or relative measure of frequency content. Examples of direct measure include a measure of magnitudes across spectral bins multiplied by a bin position of each spectral bin, an organizational analysis of the frequency content, a dominant frequency analysis, and median frequency of the absolute value of the Fourier transform. In general, direct measurements may be performed in remote monitoring networks because of computational complexity. Relative measures are based on comparisons of EGM signals before and after a frequency-analysis step that modifies signals based on their frequency content. In various embodiments, this threshold may be defined in relation to the frequency content of the conducted baseline rhythm.

Two alternate embodiments are disclosed. The first compares the EGM signal amplitude after basic filtering to the amplitude after the test step of a second high pass filter (Ratio 1).

$$Ratio 1 = \frac{Input EGM signal amplitude}{Output EGM signal amplitude}$$

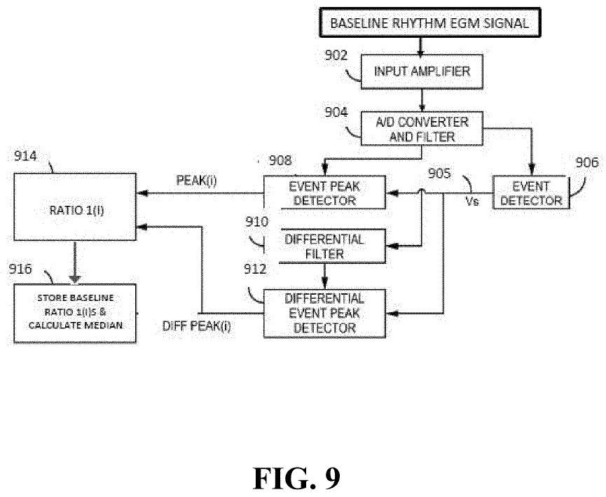

Thus, Ratio 1 is the relative amplitude of the signal before and after the frequency-analysis step. As shown in FIG. 9, in a first alternate embodiment a processor determines that an EGM signal is a high-frequency EGM signal if Ratio 1 exceeds a predetermined threshold.

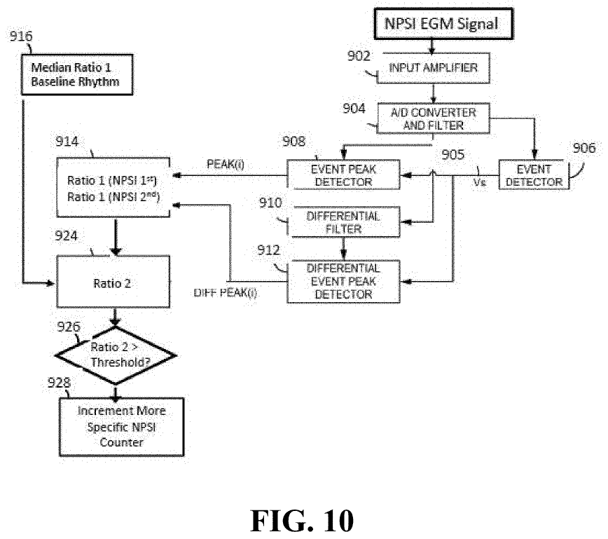

As shown in FIG. 10, in a second alternate embodiment a processor determines Ratio 1 both for EGM signals from baseline rhythm and for each NPSI EGM signal. It then determines a secondary ratio of ratios (Ratio 2), which compares Ratio 1 for each NPSI EGM signal to Ratio 1 for baseline rhythm.

$$Ratio 2 = \frac{Ratio {1}_{NPSI EGM signal}}{Ratio {1}_{Baseline rhythm EGM signal}}$$

The NPSI EGM signal is determined to be a high-frequency EGM signal if Ratio 2 exceeds a predetermined threshold.

The relative-measure frequency-analysis step measure may be a high-pass filter or a simplified surrogate for a high pass filter. One such simplified surrogate is comparison of a first-order differential signal with a threshold, as described, for example, in the context of detecting T-wave oversensing in Cao U.S. Pat. No. 9,597,525, the contents of which are hereby incorporated herein. Alternatively, the surrogate may involve comparison with a threshold of a first derivative, a second order differential signal, or a second derivative as described, for example, in Balda R, Diller G, Deardorff E, Doue J, Hsieh P. The HP ECG analysis program. Trends in computer-processed electrocardiograms 1977; 4:197-205, the contents of which are hereby incorporated herein.

For example, in an embodiment in which the frequency-analysis measure involves a differential or difference signal, a NPSI EGM signal is determined to be a high-frequency EGM if its Ratio 2 exceeds a predetermined threshold: In other words, the difference or differential operation reduces the amplitude of the NPSI EGM signal less than a prespecified fraction of the corresponding relative reduction for the patient's conducted baseline EGM signal.

In alternative embodiments, referring to FIG. 4, an initial and periodic analysis may comprise (1) determining the amplitude of a sequence of sensed EGM signals (R waves) representing a baseline rhythm after filtering and rectification by the sense amplifier; (2) applying the differential analysis to said sequence of processed baseline R waves to generate differential frequency-analysis signal for each baseline R wave; (3) for each R wave in the sequence, determining Ratio 1, the relative amplitude of the differential signal; (4) determining a measure in relation to this sequence of Ratio 1 's of baseline R-waves, such as the mean, median, mode, or modesum this example uses the median for simplicity, but it is understood that any other measure of central tendency may be used; (5) selecting a predetermined percentage of this measure (for example, 90% of the median value); and (6) storing said measure as a reference value to be included as the next RR interval (S400) for NPSI processing/determination. The information may then be processed to determine whether a NPSI is identified (S402).

When a NPSI is identified (Yes at S402), (1) the differential signal is computed for the first bounding NPSI EGM signal in the analysis window beginning the NPSI and a Ratio 1 is calculated for the first NPSI EGM signal to determine the relative amplitude of its differential signal. At S404, Ratio 1 is compared with the stored threshold value. If the first NPSI EGM signal's Ratio 1 exceeds the reference value by a predetermined amount, the EGM signal is classified with respect to frequency content as a high-frequency EGM (Yes at S404). A low-frequency EGM is classified if its Ratio 1 is less than the reference value by a predetermined amount, or neither. This process is then repeated for the second bounding NPSI EGM signal in the analysis window corresponding to the end of the NPSI. In embodiments, if a determination is made that a high-frequency EGM is not classified (no at S404), a count may be maintained and incremented that indicates the number of NPSIs (S414) not considered high-frequency NPSIs. The processes may then be repeated for next RR intervals (S400). If it is determined that the NPSI is a high-frequency EGM (yes at S404), a count may be maintained and incremented that indicates the number of More Specific NPSIs (S408). If either bounding EGM corresponds to a ventricular paced event, then only the un-paced EGM is analyzed. In patients who are pacemaker dependent and have no analyzable baseline rhythm other criteria must be used. In one embodiment, Ratio 1 for one of the NPSI EGM signals must exceed a fixed percentage. This fixed percentage may be calculated, for example, from the average of the 90% value of Ratio 1 for a population of patients who have a conducted baseline rhythm. In another embodiment, the frequency-analysis step may comprise a high-pass filter with a cut off in the range of 25-40 Hz.

In embodiments, an alert for a lead system condition may be triggered (S412) if the count of More Specific NPSIs exceeds a threshold value in a predetermined time period (S410). Typical values might be a count of 10 to 30 in one to three days.

Referring to FIGS. 9 and 10, embodiments of schematic flow charts of relative-measure frequency-analysis will be described. The apparatus and techniques represented by diagram 900 pertain to the primary EGM sensing channel. Input amplifier 902 amplifies the input EGM signal developed across the sensing electrodes coupled to the sensing channel and passes the signal to an A/D (analogue to digital) converter and filter 904, and an event detector 906.

FIG. 9 illustrates processing of a series of sensed baseline-rhythm EGM signals, and FIG. 10 illustrates processing of the two EGM signals corresponding to the NPSI. The amplified EGM signal from input amplifier 902 is converted to a digital signal by A/D converter 904 and digitally filtered by bandpass filter 904. In various embodiments, the bandpass filter may have a pass band of 10 to 40 Hz. The filtered digital signal is provided to cardiac event detector 906 that uses a dynamic R-wave sensing threshold set based on the peak amplitude of the last sensed event. When the dynamic sensing threshold is crossed, a V sense (Vs) event signal 905 is passed from an electrical sensing module to a cardiac signal analyzer.

In various embodiments, a cardiac signal analyzer receives Vs event signal 905 and the filtered digital signal from A/D converter and filter 904. In embodiments, a cardiac signal analyzer includes an event peak detector 908, a differential filter 910, and a differential signal event peak detector 912. The event peak detector 908 samples the filtered digital signal before and after each Vs event signal. In one example, the filtered digital signal is sampled for 15-20 ms before the Vs event signal and 100 ms after the Vs event signal at a sampling frequency of 256 Hz to 1024 Hz by event peak detector 908. In embodiments, a maximum signal amplitude of the samples is identified as the peak amplitude of the sensed event. The peak amplitude of each i^{th} baseline event (PEAK(i)) in the event window is provided as input to ratio analyzer 914.

In embodiments, the filtered digital signal from A/D converter and filter 904 is also provided to a differential filter 910. Differential filter 910 determines a first order differential signal, e.g., as given by Y(i)=x(i)−x(i−b) where x(i) is the current sample point amplitude and x(i−b) is the amplitude of a signal sample point that precedes the current sample by b measurements. In embodiments, b=1. In other examples another, form of high pass or band pass filter may be used to obtain a high-pass filtered signal of the filtered digital signal output from the A/D converter and digital filter 904.

In embodiments, the differential filtered signal is passed to a differential event peak detector 912, which samples the differential filtered signal before and after the Vs event signal 905 and determines the differential signal peak amplitude, DIFF PEAK(i), of each sensed event during a series of sensed baseline events. The differential filtered signal may be sampled for 15-20 ms before the Vs event signal 905 and 100 ms after the Vs event signal at a sampling rate of 256 Hz to 1024 Hz. The differential peak amplitude DIFF PEAK(i) of each Vs event is provided as input to NPSI processor comprising blocks 914, 916, 924, 926, and 928. NPSI processing block 914 computes Ratio 1 for each baseline rhythm EGM signal by comparing the output differential signal event peak amplitude from block 912 to the peak amplitude of the filtered signal from block 908. In some embodiments, R1 ratios for a series of baseline-rhythm EGM signals are stored at baseline EGM processing block 916, and a median of these ratios is calculated for use in the relative-measure frequency analysis.

With additional reference to FIG. 10, when a NPSI occurs, the EGM signal corresponding its two sensed events are similarly processed by steps 902-914. Then in step 924 Ratio 2 is computed for both the first and second sensed events of the NPSI, using the corresponding Ratio 1 ' s from step 914 and the median Ratio 1 in baseline rhythm from step 916. In step 926 Ratio 2, for the NPSI's first and second sensed events are compared with one or more predetermined thresholds. In embodiments, if Ratio 2 for either of the NPSI's sensed events exceeds a threshold that is greater than or equal to 1, the More Specific NPSI counter is incremented in step 928. In other embodiments, if both of the NPSI's sensed events exceeds a threshold that is less than 1, the More Specific NPSI counter is incremented. In general, the high pass filtering effect of the differential filter 910 attenuates oversensed cardiac signals (e.g., ventricular fibrillation, P waves, T waves) more than oversensing caused by lead system conditions. Thus, the relative difference between their differential peak amplitudes will be greater than the relative difference between the peak amplitudes of the filtered signals. In general Ratio 2 for each of the NPSI's sensed events will be substantially less than 1 if the sensed event is an oversensed cardiac signal, greater than or equal to 1 if the sensed event is caused by oversensing from a lead system conditions, and close to 1 if the sensed event is a baseline rhythm EGM signal.

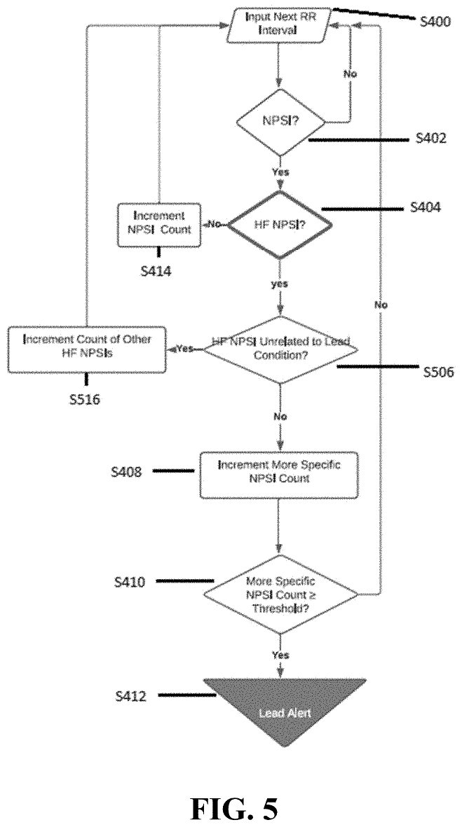

Reject High-Frequency Short Intervals Caused by Conditions Other than Lead Failure Referring to FIG. 5, an additional criterion, with reference to the flowchart diagram of FIG. 4, may be added to the definition of More Specific NPSI, which rejects a high-frequency NPSI known to have specific causes other than lead failure (S506). The most common cause of false-positive oversensing alerts caused by high-frequency NPSIs is external EMI. A less common cause is diaphragmatic myopotentials. These signals are almost always repetitive, so this analysis is applied only if a threshold number of high-frequency NPSI's occur in a short period of time (e.g. 6 in 2 s, 12 in 3 s). It is understood that this embodiment may be implemented only to reject EMI or to reject both diaphragmatic myopotentials and EMI.

Pacemakers and ICDs use multiple methods to reject EMI, for example Zhang (U.S. Pat. No. 10,583,306 B2), the disclosure of which is hereby incorporated by reference herein, teaches use of a notch filters at the electrical mains frequency (50-60 Hz) to reject EMI.

An alternative approach that applies to dedicated-bipolar sensing is to classify NPSIs based on the observation that external EMI is recorded on the far-field (shock) channel with relative amplitude (vs. baseline R wave) greater than signals on sensing channel. So external EMI should cause simultaneous signals on both sensing and far-field channels with higher amplitude on the far-field channel. One approach includes these steps:

(1) If a threshold number of high-frequency NPSIs occur in a short period of time (e.g. 6 in 2 s, 12 in 3 s), check the interval on the far-field channel around the two sensed events that define the NPSI on the sensing channel. (2) Choose a narrow window, ≤±50 ms around each of the two sensing channel events. (3) Do not classify the interval as a More Specific NPSIs if a far-field EGM (see Gunderson U.S. Pat. No. 8,792,971 B2 the disclosure of which is hereby incorporated by reference herein) corresponds to each of the NPSI's EGMs on the sensing channel and both far-field EGMs are sufficiently large. The criterion used may be applied only to the far-field channel, for example an absolute threshold (e.g. ≥1 mV) and/or a relative threshold (e.g. ≥25-50% of baseline far-field R-wave amplitude). Alternatively, the criterion may require computing the amplitude of each far-field and near-field signal in the analysis windows of the EGMs bounding the NPSI as well as a measure (e.g. median) of baseline R-wave amplitude. Then, of the two EGMs bounding the NPSI on each channel, the one with far-field R-wave amplitude closest to the baseline R-wave amplitude is discarded, and the ratio of the other two is calculated (far-field R wave amplitude/near-field R wave amplitude). This ratio must be greater than a threshold (likely between 0.5 and 1.5 5) for the NPSI to be considered as a potential EMI signal. The signal is also considered as a potential EMI signal if the far-field signal saturates the amplifier and is too large to be measured. Because this analysis is not performed unless NPSIs are repetitive, a single high-frequency NPSI with signals on both the near-field and far-field channels is not rejected. Such signals may represent an inner insulation breach that causes contact between the ring-electrode cable and RV shock coil.

Diaphragmatic myopotential signals represent an uncommon source of false-positive oversensing alerts caused by high-frequency NPSIs. Diaphragmatic myopotentials are low-amplitude and repetitive signals with higher frequency than cardiac EGMs. Oversensed diaphragmatic myopotentials typically have amplitudes only slightly above the dynamic sensing threshold. In contrast, some oversensed signals from lead failure usually have larger amplitude. Oversensing of diaphragmatic myopotentials is rare with dedicated-bipolar sensing. The principal uses of this component embodiment apply to integrated-bipolar sensing and dedicated-bipolar sensing when specific low-frequency attenuation filters are used. In embodiments, it could be programmed on as an optional feature if diaphragmatic myopotentials are oversensed. In one embodiment, a high-frequency NPSI is classified as More Specific NPSI only if the amplitude of one bounding EGMs exceeds a threshold (e.g., ≤0.7 mV). It is understood that other criteria may be developed to identify and reject non-lead related causes of high-frequency NPSIs.

If the determination is made that the NPSI signal is unrelated to a lead condition (i.e., yes at S506), a count may be maintained and incremented to reflect the number of high-frequency NPSIs other than those reflecting More Specific NPSI condition (S516). If the determination is made that the NPSI signal is related to a lead condition (i.e., no at S506), a count may be maintained and incremented to reflect the number of high-frequency NPSIs that reflect More Specific NPSI condition (S408). Thus, further defining and tracking high-frequency NPSIs determined to have specific causes of lead failure. In embodiments, an alert for a lead system condition may be triggered (S412) if the count of More Specific NPSIs exceeds a threshold value in a predetermined time period (S410). Typical values might be a count of 10 to 30 in one to three days.

Lead Alert Before a Threshold Count of More Specific NPSIs is Reached if an Additional Specific Criterion is Met

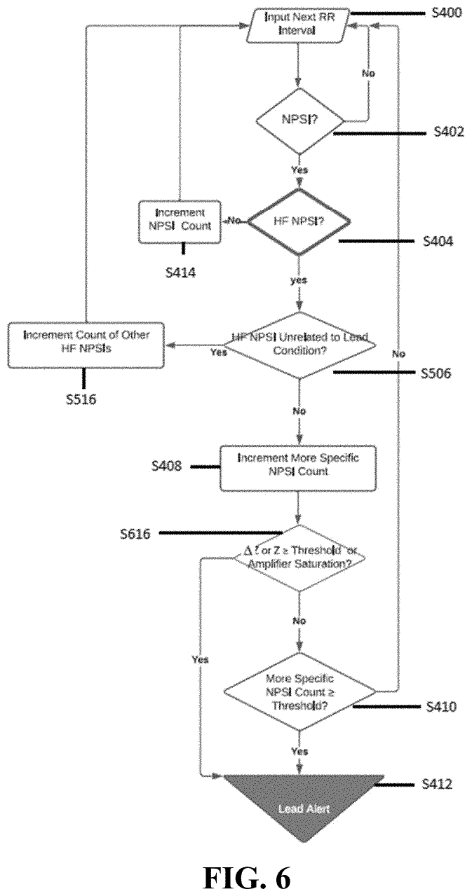

In embodiments, referring to FIG. 6, an additional step may be added to embodiments of the flowchart diagrams, as described herein, to alert before a threshold count of More Specific NPSIs is reached if other features specific for a lead system condition are met (yes at S616). One or multiple specific criterion may be added. The description of this embodiment provides two illustrative examples, but it is understood the embodiments described may include any criterion known in the art that increases specificity of More Specific NPSIs for a lead system condition. One criterion is based on the knowledge that high-frequency EGM signals that saturate the sense amplifier have high specificity for conductor fracture or header-connection problems if EMI is excluded. The second example criterion is based on the knowledge that the combination of a More Specific NPSI and abnormal impedance measurement has high specificity for lead system condition. Note, if a threshold count of More Specific NPSIs is not reached (e.g., no at S616), the process may proceed as previously discussed herein (to S410).

### Example 1. Either Bounding EGM Saturates the Sense Amplifier (Δ)

An alert and storage of an EGM may be conducted if any signal in a sensing-channel analysis window that begins or ends a More Specific NPSI (1) has as amplitude equal to the sense amplifier's maximum filtered value, and (2) exceeds baseline R-wave amplitude by a relative or absolute threshold (e.g., percentage or value in mV). The baseline R-wave amplitude may be determined by various methods. For example, take the last 8-12 R-wave amplitude measurements. Chose the 2^{nd} or 3^{rd} largest.

### Example 2. An Impedance or Impedance-Variability Measurement Triggered by a More Specific NPSI is Abnormal (Z)

V-V intervals are measured continuously, but impedance is measured intermittently. The same lead motion that causes oversensing signals to occur in conductor fracture or insulation breach causes simultaneous changes in pacing impedance. The occurrence of one or more of the More Specific NPSI events may be considered a candidate surrogate for abnormal impedance. Thus, in an implanted system with a lead failure, an impedance measurement simultaneous with a More Specific NPSI has a higher likelihood of being abnormal than an impedance measurement made at a predetermined time. Additionally, when impedance measurements are made in quick succession, it is likely that measurement variability of an intact lead will be much lower than with the conventional approach of measurements separated by ≥1 h, as performed presently by all manufacturers. Thus, a sequence of impedance measurements triggered by a single More Specific NPSI is likely to have minimal variability in an intact lead, is likely to have significant variability in a pace-sense lead failure. In conductor fracture, sequential measurements are likely to occur with fracture faces in different degrees of contact; in in insulation breaches, sequential measurements are likely to occur with differing degrees of metal-to-metal contact.

An impedance or impedance-variability measurement is triggered by the occurrence of one or multiple More Specific NPSIs. The criteria for initiating an impedance or impedance-variability measurement may depend on the embodiment. Examples include: (1) any More Specific NPSI, (2) The first More Specific NPSI if none have occurred for a predetermined period (e.g. 1-30 days), or multiple More Specific NPSIs in a short time (e.g., 3 in 5 minutes). More generally, a measurement may be triggered by increase in frequency of More Specific NPSIs or a decrease in time between More Specific NPSIs.

Once the trigger for impedance measurement is satisfied, a rapid sequence (“train”) of subthreshold pulses is delivered to measure impedance (e.g., 10-100 pulses at 10-100 ms intervals). Thresholds for impedance alerts may be based on absolute, impedance values, abrupt absolute changes in impedance, or abrupt relative changes in impedance are well known in the art. Additionally, various threshold criteria may be used for an impedance-variability alert. One embodiment is an impedance-variability threshold based on the difference between maximum and minimum impedance values determined by the train of pulses (ΔZ=Zₘₐₓ−Zₘᵢₙ). Another embodiment may first exclude the maximum and minimum impedance values as potential outliers and then be based on the difference between maximum and minimum values of the remaining measurements. Candidate values for the ΔZ threshold are in the range 2-50Ω with a preferred value likely in the range of 3-10Ω. This ΔZ threshold differs from any previously used impedance threshold both because the “baseline” is determined concurrently and the difference is determined as an absolute, rather than relative difference.

Gunderson (U.S. Pat. No. 9,522,277 B2), the disclosure of which is hereby incorporated by reference herein, teaches a related concept in which a single impedance measurement of one or more leads is triggered by signal saturation, and an alert is triggered for an impedance value that exceeds a threshold. The embodiments disclosed herein differ from that of Gunderson ′277 by triggering a rapid sequence of impedance measurements immediately following the second EGM of a More Specific NPSI (whether or not saturation occurs) and triggering an alert if the variability in the sequence of measurements exceeds a threshold (rather than if the impedance value exceeds a threshold).

Determine if there is Threshold Count of Other NPSIs to Trigger a More General Oversensing Alert

Lead alerts should receive the highest priority and urgency. Alerts for other causes of oversensing may be useful but should usually be delivered with lower priority/urgency or be logged as “observations” rather than trigger an alert.

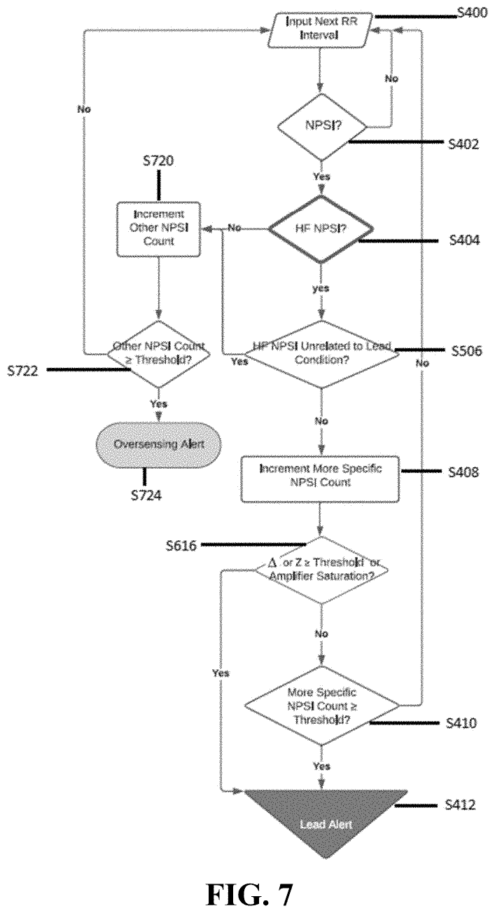

Referring to FIG. 7, in embodiments, a determination may be made if there is a sufficient count of NSPIs unlikely to be related to a lead system condition in a pre-specified time interval (or increase in their baseline frequency). In embodiments of the flowchart diagrams, as described herein, depict a simpler embodiment that utilizes a count of NPSIs that are not More Specific NPSIs (“other NPSIs”) (S720) and triggers an alert or observation (S724) if a threshold count is reached in a predetermined time interval (S722). In embodiments, if this threshold is reached, an Oversensing Alert is triggered (S724). Note that an “Oversensing Alert” is triggered rather than a “Non-Lead Oversensing Alert”, because of the unlikely possibility that lead failure may trigger such an alert. The limitation of this simpler embodiment is that it does not diagnose the cause of oversensing. In embodiments, the number of “other NPSIs” may be maintained and incremented (S720) based on determinations made regarding whether or not the NPSIs are high-frequency NPSIs (no at S404) and/or whether the classified high-frequency NPSIs are unrelated to lead conditions (yes at S506).

However, alternate embodiments could also distinguish among various types of NPSIs that are not More Specific on the basis of frequency. In such an embodiment, the specific threshold count and time frame for an alert may depend on the frequency content of oversensed signals and whether or not oversensing is repetitive. For example, the threshold might be a count of 5 to 10 in 1 minute to alert for frequent oversensing vs. a count of 20 to 50 low-frequency NPSIs over a longer period (e.g., 1 to 3 days), since low-frequency NPSIs usually represent oversensing of physiologic signals, and occasional oversensing of these signals may not warrant an alert.

Determine if Low-Frequency NPSIs are Caused by Known Types of Cardiac Oversensing.

Specific interval and EGM patterns around low-frequency NPSIs usually indicate the cause of cardiac oversensing (Swerdlow C D, Asirvatham S J, Ellenbogen K A, Friedman P A.

Troubleshooting implanted cardioverter defibrillator sensing problems I. Circulation Arrhythmia and electrophysiology December 2014; 7:1237-1261.) The need for troubleshooting and the specific troubleshooting approach differs for different causes of low-frequency NPSIs. In embodiments, the steps, as described in reference to FIG. 7, may identify low-frequency NPSIs caused by sensing or oversensing of biologic, cardiac EGMs. The examples given are illustrative only, and it is understood that other methods may be used to classify low-frequency NPSIs.

Classify NPSIs as ventricular fibrillation NPSI if the baseline ventricular cycle length (baseline cycle length) indicates ventricular fibrillation. Ventricular fibrillation is a common cause of short V-V intervals, and some successive EGMs in ventricular fibrillation may be short enough to meet criteria for NPSIs. The baseline cycle length may be calculated in various ways used in ICDs. For example, mean or median of last 4-12 intervals<threshold cycle length. For the first index NPSI identified in a time period (e.g., 30 s) or number of intervals (e.g., 50 intervals), determine the baseline cycle length. It is definitional that no baseline interval will be an NPSI. If the baseline cycle length is shorter than a threshold (in one embodiment, range for threshold 250-320 ms), then the index NPSI interval is considered a ventricular fibrillation interval. Importantly, this analysis only applies to the first NPSI in a given sequence of NPSIs, since the mean or median interval may become short rapidly once repetitive, fracture-induced oversensing begins. If a NPSI is determined to be a ventricular fibrillation interval, then a method needs to be implemented to prevent future NPSIs in the same clinical ventricular fibrillation episode from triggering an alert. One method is to use the same baseline cycle length preceding the first ventricular fibrillation interval until no NPSIs are recorded for a given time period (e.g., 10 to 30 s) or a corresponding number of intervals. Alternatively, analysis of NPSIs is suspended until a sufficient number of consecutive (e.g., 2 to 8) or closely spaced intervals (e.g., 2 of 3, 4 of 5) are in the sinus zone.

Identify P-wave or T-wave Oversensing. These types of physiologic oversensing result in characteristic patterns of alternating intervals and “R” wave amplitude unlikely to occur in lead failure. See for example, Swerdlow C D, Asirvatham SJ, Ellenbogen K A, Friedman P A. Troubleshooting implanted cardioverter defibrillator sensing problems I. Circulation Arrhythmia and Electrophysiology 2014; 7:1237-1261 or Gunderson U.S. Pat. No. 7,783,354. In one embodiment, the following criteria must be met: (1) The baseline cycle length (as defined previously) preceding the first NPSI is in the sinus zone and/or longer than a cycle-length threshold (e.g., 400 or 500 ms). (2) None of the EGM signals in baseline rhythm (corresponding to the sensed events used to determine baseline cycle length) has amplitude exceeding the median by more than an absolute (e.g., 5 mV) or relative (e.g., 50%) measure, since conductor fracture signals are often large. (3) Two to four of 8 consecutive intervals are NPSIs. More generally N/2, (N−1)/2, or (N−2)/2 of N consecutive intervals are NPSIs, where N is in the range 6-20. (4) The measured amplitude of the NPSI's low-frequency EGM signal is below a threshold after initial bandpass filtering. That threshold that may depend on baseline R-wave amplitude (e.g., 25% if ≥3 mV, 50% if ≤3 mV). (5) Either the interval preceding or following the NPSI is close to the value expected if the NPSI represented P or T wave oversensing (baseline cycle length-NPSI).

Gunderson (U.S. Pat. No. 7,783,354), the disclosure of which is hereby incorporated by reference herein, teaches a method to identify different causes of oversensing using the temporal pattern of inter-EGM intervals, which h is hereby incorporated by reference herein, teaches a similar method for identifying intracardiac oversensing that may also be incorporated in this embodiment as an alternative. It is also understood that this analysis could be performed in a remote monitoring network using machine learning.

Perform Additional Analysis of Low-Frequency NPSIs without a Known Non-Lead Cause

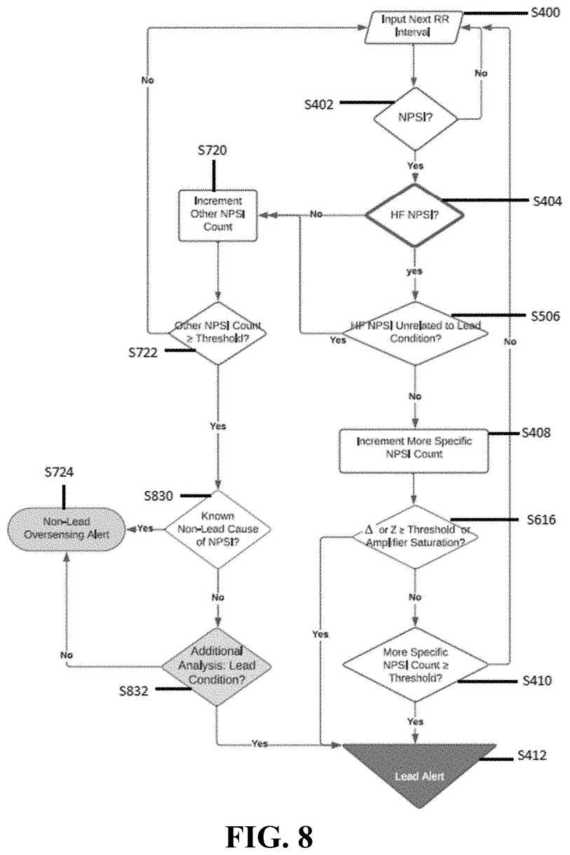

Rarely are lead failures present with NPSIs, but none or an insufficient number are More Specific NPSIs. Referring to FIG. 8, additional step(s) may be added to embodiments of the flowchart diagrams, as described herein. If known causes of non-lead system related NPSIs have been rejected (no at S830), then a lead failure may be present. In this step, additional analysis is performed (S832) if a threshold count of other NPSIs is reached (S722) and no specific cause is identified (no at S830). If a lead system condition is identified (yes at S832), a Lead Alert is triggered (S412). Otherwise, an optional, lower-priority Non Lead Oversensing Alert or Observation is triggered (S724).

Various additional analyses are possible, either individually or combined. Two illustrative examples are considered, but these are not intended to be limiting. In one embodiment, an impedance or impedance-variability analysis described in FIG. 6 is performed. The measurement is triggered immediately after the first other NPSIs without a known non-lead system related cause after a threshold count of such NPSIs is reached. A Lead Alert is triggered if the impedance or impedance-variability measurement is abnormal. In another embodiment, the count of More Specific NPSIs required to trigger a Lead Alert is reduced if a threshold count of other NPSIs without a known non-lead system related cause is reached. Alternatively, a Lead Alert may be triggered by a combined count of More Specific NPSIs and non-More Specific NPSIs without a known cause.

### Example Clinical EGMS for Lead Fracture Versus NPSI for Other Reasons

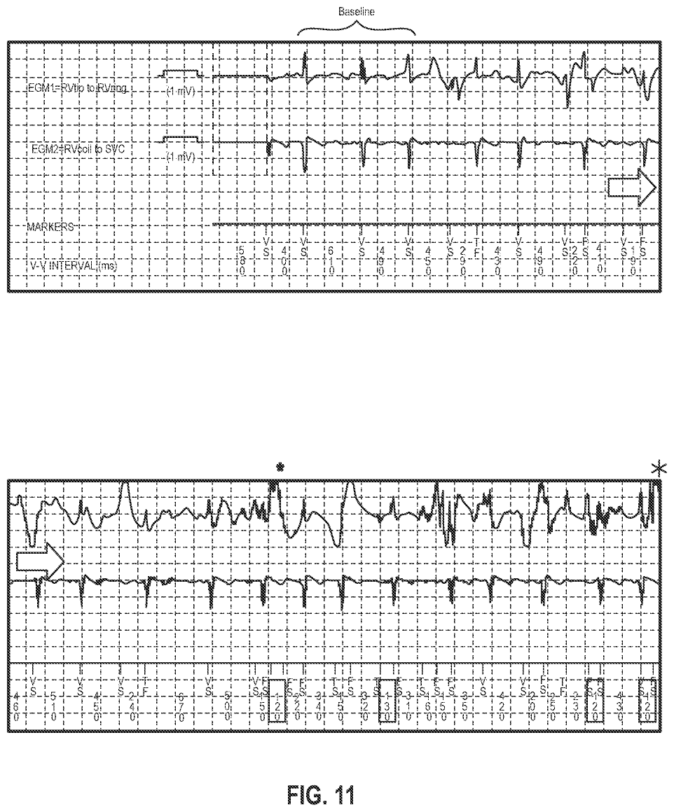

FIG. 11 shows an embodiment of clinical EGM data for a conductor fracture. Upper and lower panels are continuous. In order, RV pace-sense channel, a shock channel, and ventricular marker channel are shown with device sensed RR intervals displayed. Black box(es) on the marker channel indicate NPSIs. Oversensed signals beginning and ending NPSIs have slope (frequency content) similar to or greater than that of baseline rhythm R waves (coinciding with R waves on shock channel). Asterisks denote amplifier saturation during signals bounding NPSIs.

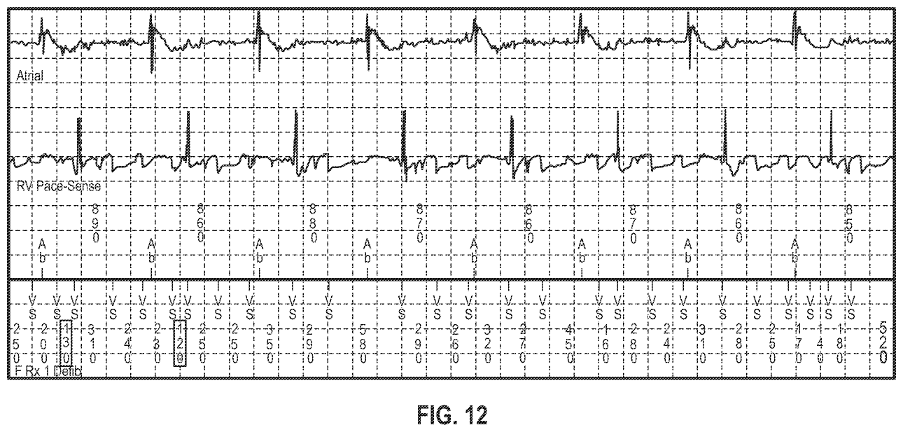

FIG. 12 shows an embodiment of clinical EGM data for a conductor fracture. In order, atrial channel, RV pace-sense channel, and ventricular marker channel are shown with device sensed RR intervals displayed. Boxes on marker channel indicate NPSIs. Even though they have low amplitude, oversensed signals beginning and ending NPSIs have slope (frequency content) similar to or greater than that of baseline rhythm R waves (coinciding with R waves on shock channel).

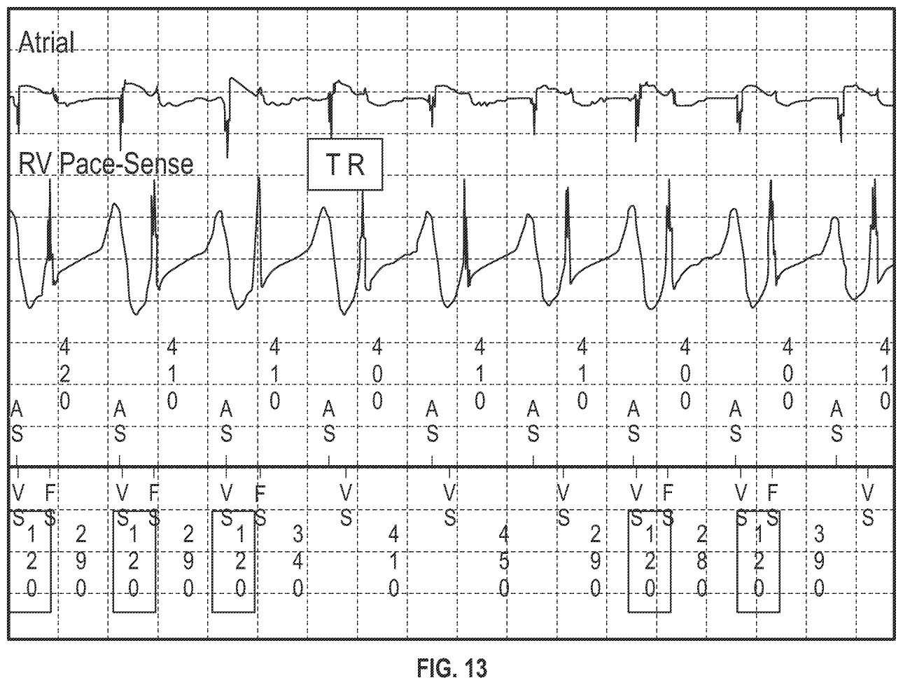

FIG. 13 shows an embodiment of clinical EGM data for NPSIs caused by T-wave oversensing comprising the same channels and abbreviations as in FIG. 12. Each NPSI begins with a wide, rounded (low-frequency) T wave and ends with a higher frequency baseline R wave.

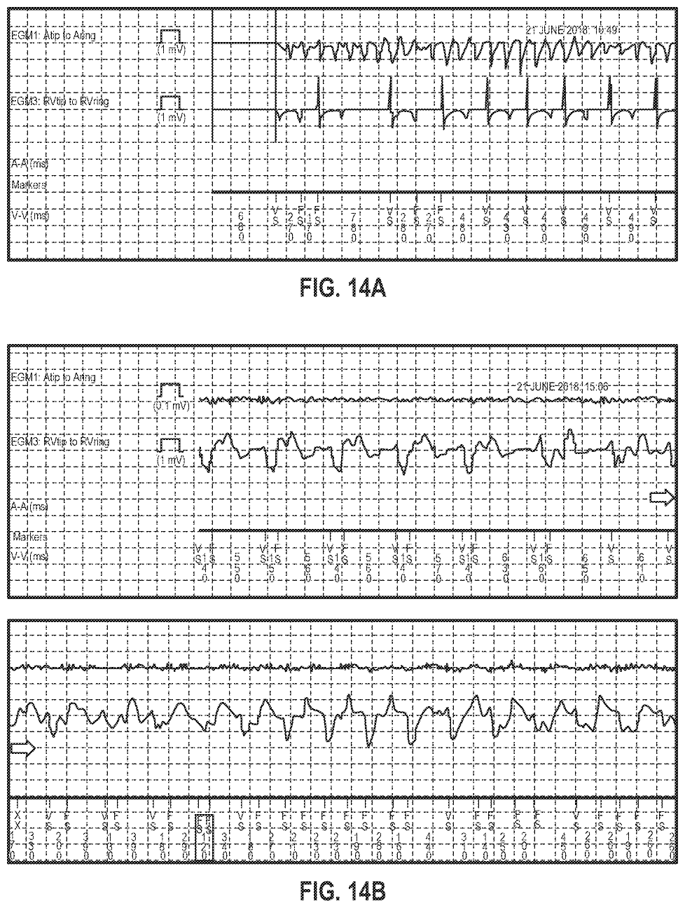

FIG. 14 shows an embodiment of clinical EGM data for NPSIs caused by R-wave double-counting comprising the same channels and abbreviations as in FIG. 12. Panel A. shows baseline rhythm with sinus rhythm R waves. Panel B shows R-wave double-counting. Box denotes NPSI. Oversensed signals are wider and have lower amplitude than sinus rhythm signals, indicating that oversensed signals have lower frequency content.

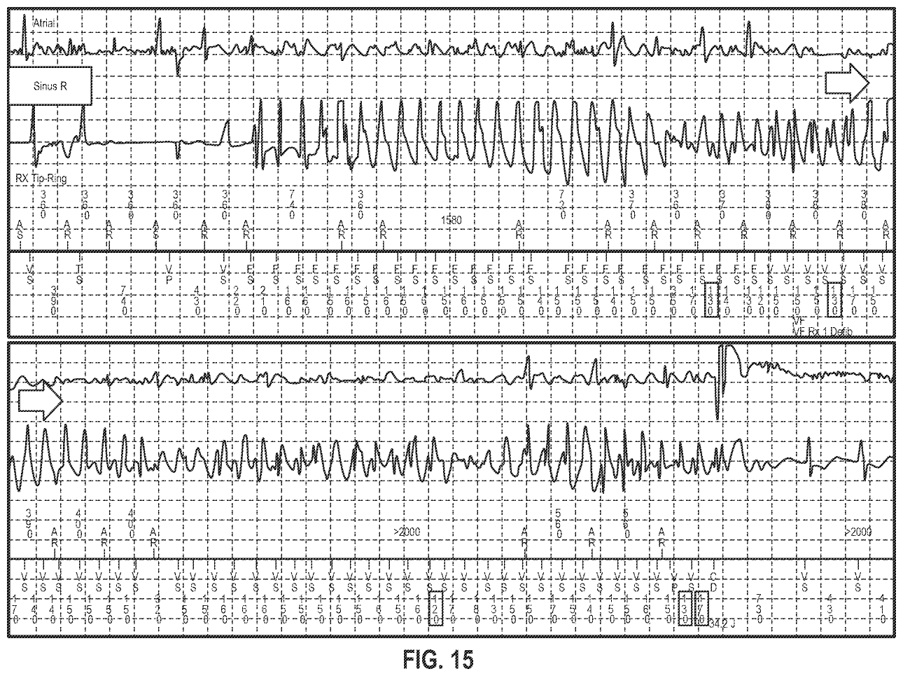

FIG. 15 shows an embodiment of clinical EGM data for NPSIs caused by EGM signals in ventricular fibrillation comprising the same channels and abbreviations as in FIG. 12. The upper and lower panels are continuous. The first EGM signal in the upper panel is a conducted sinus beat. EGMs corresponding to NPSIs denoted by boxes are wider than baseline, sinus-rhythm R wave, indicating lower frequency.

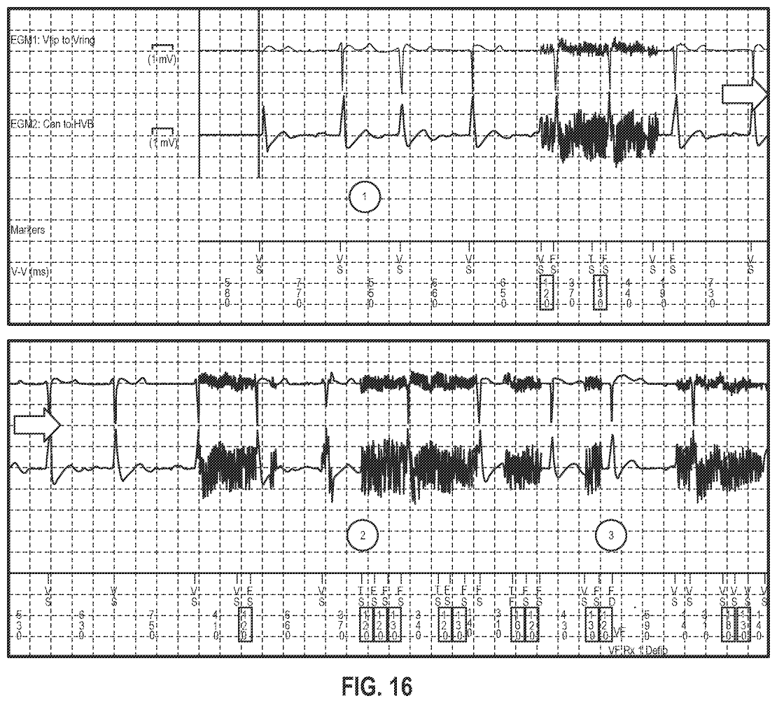

FIG. 16 shows an embodiment of clinical EGM data for NPSIs caused by electromagnetic interference. The panels are continuous. Oversensed signals beginning and ending NPSIs have high frequency and higher amplitude on far-field (Can to HVB) channel than pace-sense channel (Vtip to Vring).

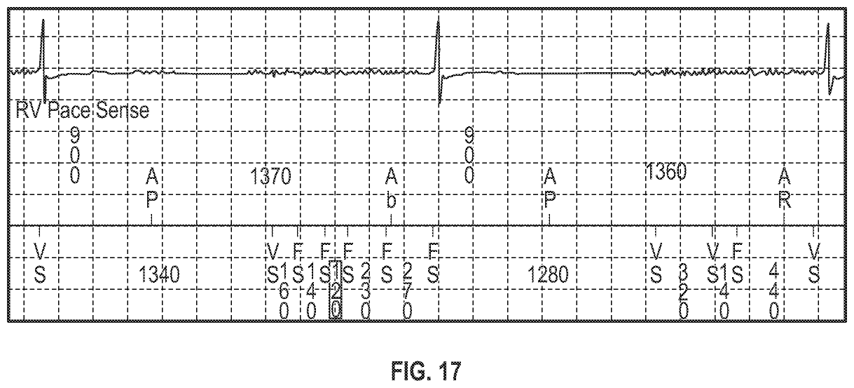

FIG. 17 shows an embodiment of clinical EGM data for NPSIs caused by oversensing of diaphragmatic myopotentials. Rapid oversensed signals including both EGM signals corresponding to NPSI are denoted by black box(s) and have extremely low amplitude.

Threshold Counts for Alerts

It is understood that, in various embodiments alerts may be configured for any threshold number of short-interval events≥1 in any predefined period of time.

General Considerations

The threshold number and relevant time period may be specific to the criterion or criteria met.

To prevent inappropriate shocks, this time period will be short for thresholds that trigger lead alerts, especially if conductor fracture is suspected. This is based on the observation that early-stage, conductor fracture-related oversensing usually clusters in periods<3 min. However, short-interval events may be more sporadic in some insulation breaches, so the threshold event number and time interval may depend on the relative risk of fracture vs. breach for an individual lead.

Various embodiments may be considered. The list below provides examples, but is not intended to be limiting:
In one embodiment, it may require a count of 1 to 5 More Specific NPSIs in a short time window (30 s-3 min). Or it may require that this count be satisfied multiple times (preferred 2 to 4 times) in 24 hours, each separated by at least 5 minutes.
A second embodiment alerts for the first More Specific NPSI in a pre-specified period (e.g., 7-30 days, etc.)
In a third embodiment, a Lead Alert may be triggered by a single More Specific NPSI that also satisfies one additional criterion as described in Embodiment 3 (ΔZ or saturation criteria).

Threshold Selection in Relation to Baseline Frequency of Events

Lead failures rarely occur early after new lead implant (although they can occur early after generator change). NPSIs caused by non-physiologic combinations of physiologic signals often occur in isolation when analyzed over short time frames (e.g., 1 to 5 min) or at an approximately constant rate when analyzed over longer periods (e.g., 1 to 30 days). The same frequency of NPSIs is more likely to indicate lead failure if there is an abrupt increase in frequency than if the frequency is constant over time.

In embodiments, the first 30-90 days after lead implant may be used to determine a baseline frequency of More Specific NPSIs (e.g., different for integrated-bipolar vs. dedicated-bipolar leads). The time interval for analysis may vary (e.g., 1 h to 7 days, most likely 12-48 h). The maximum number of More Specific NPSIs in a short during the baseline period will also be determined (e.g., 5 s, 15 s 30 s, 1 min, 3 min). In alternative embodiments. a measure of the time between More Specific NPSIs may be used if there are a sufficient number of events (e.g., ≥5). For example, the median, mode, or modesum time between events may be used instead of frequency. In this embodiment, the threshold for Lead Alert is determined by a measure of increase from baseline frequency (More Specific NPSIs per unit time) or decrease in time between events. Various criteria are possible. They may be used alone or in combination. For example: a pre-specified percentage increase in frequency of events over a fixed time interval relative to baseline or a pre-specified percentage decrease in time between events.

Additional Considerations

Application to Sequences of Short Intervals (Nonsustained Tachycardias)

CIEDs store sequences of short intervals as “nonsustained tachycardias.” Various embodiments as disclosed herein apply to individual non-physiologic short. However, embodiments of the disclosure may also be applied to a sequence of intervals with a relatively short mean or median cycle length (e.g., 150-300 ms). In this case, the sequence will be non-physiologic short sequence if any interval in the sequence is a More Specific NPSI.

Adjudication Rules

It is anticipated that there will be discrepancies in classifying sequential NPSIs. Adjudication rules may be required because short intervals occurring in succession likely have the same root cause. The list of examples below are offered as illustrative of adjudication rules rather than intended to be limiting. It is understood that other adjudication rules may also be necessary.
If a lead alert is triggered, any temporally-related, NPSI alert will be suppressed. For example, non-lead oversensing alerts will be suppressed if triggered from 5 min before the lead alert until in-person interrogation or remote re-programming.
Alternatively, the decision to trigger an Oversensing or Non-Lead Oversensing alert may suppressed if the count of More Specific NPSIs is close to threshold. For example, if the count exceeds 70% of threshold, then a Lead Alert is triggered instead.

Additional Consequences of Short Intervals and Alerts

Stored EGMs

In embodiments, short interval events may trigger the storage of an EGM. The first NPSI of any type (e.g., More Specific NPSI, other NPSI) will trigger storage of an EGM. In embodiments, the number of stored EGMs allocated to each type of short-interval event may vary, depending on event priority (e.g., store a greater number of More Specific NPSI events than other NSPI events) and other utilization of memory. The priority for storage of events may be determined in various ways, for example, the first event after a prespecified time for each type may be retained, otherwise a first-in, first-out concept may be implemented. The strategy for storing alert EGMs will be similar to that for storing EGMs for isolated short-interval events, except that alert events will take priority.

Disabling Alerts

Analyses of NPSIs may be disabled after a lead alert until programmer interrogation or remote programmer interrogation/programming is performed.

Remote Monitoring

For simplicity, various embodiments are described with all steps performed in the implantable medical device. However, it is understood that some or all steps may be performed in a remote monitoring network. Alerts may be direct to the patient, in the remote monitoring network, or both. CIED-generated alerts permit direct patient notification independent of remote monitoring network connectively. But remote monitoring networks have substantially greater computing power and memory than implantable medical device, and network computing is not constrained by power consumption as are implanted microprocessors. In another embodiment, the analysis for alerts may be performed only in the remote monitoring network. Embodiments 4 and 5 that classify the root causes of NPSIs may be particularly suitable to remote monitoring analysis based on algorithms or machine learning. More generally, it may be desirable to perform a preliminary analysis (e.g., FIGS. 4-6) in the implantable medical device and optional higher-level analyses (e.g., FIGS. 7-8) in the remote monitoring network.

In one embodiment, the automated method is performed in near real time as NPSIs occur by the implanted device (e.g., on the order milliseconds to seconds). In another embodiment, the automated method is performed in a periodic “batch” mode where the analysis is performed by an external device in response to interrogation or periodic downloading of data from the implanted device. In another embodiment, the primary analysis comprising essential steps or essential and recommended steps performed in the implantable medical device and optional higher-level analyses are performed in a batch mode in the remote monitoring network. Examples of this kind of graduated analysis performed by different automated processors is described, for example, in U.S. Pat. No. 8,401,644 B2, the disclosure of which is hereby incorporated by reference herein. In this embodiment, there is a basic level of monitoring based on the first analysis even if patients lose connectivity to the remote monitoring network, for example, because they go on vacation without their monitor, or are out of cell phone service range.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. An automated method of identifying a lead system condition of an implantable lead system operably positioned to transmit signals from one or more chambers of the heart of a patient and operably coupled to a cardiac implantable electrical device for sensing that is operably coupled to the implantable lead system, the automated method comprising:
sensing an electrogram signal from an electrode pair in which at least one electrode is on the implantable lead system;
evaluating the electrogram signal for a series of sensed events;
for each interval between successive sensed events, determining whether that interval is a non-physiological short interval (NPSI), and if so:
using a processor system to analyze if the NPSI is a more-specific NPSI based on a measure of the frequency content of the electrogram signals in the two analysis windows corresponding to the sensed events that begin and end the NPSI; and
once a predetermined number of more-specific NPSIs have been identified within a predetermined monitoring time period, generating a lead system condition alert.

2. The automated method of claim 1 wherein an electrogram signal in an analysis window is determined to be a high-frequency electrogram signal if a measure of a frequency content of the electrogram signal in the analysis window exceeds a first predetermined threshold and a low-frequency electrogram signal if a measure of a frequency content of the electrogram signal in the analysis window is less than a second predetermined threshold.

3. The automated method of claim 2 in which a NPSI is analyzed to be a more-specific NPSI if:
at least one of the electrogram signals in the two analysis windows corresponding to the sensed events that begin and end of the NPSI is a high-frequency electrogram; or
both of the electrogram signals corresponding to the sensed events that begin and end the NPSI are not low-frequency electrograms.

4. The automated method of claim 3 in which a NPSI is analyzed to be a more-specific NPSI only if:
further processing is performed on both electrogram signals corresponding to the two analysis windows, with or without processing of additional electrogram signals and related intervals; and
this processing does not identify a cause of the NPSI unrelated to a lead system condition.

5. The automated method of claim 4 in which the additional electrogram signals and related intervals correspond temporally to the two analysis windows of the NPSI and are recorded from one or more electrode pairs on one or more conductors in the lead system that are different from the electrode pair that is sensing the electrogram signal.

6. The automated method of claim 4 in which the additional electrogram signals and related intervals correspond to the two analysis windows recorded from the electrode pair that is sensing the electrogram signal immediately before or after the NPSI.

7. The automated method of claim 2 wherein the first predetermined threshold and the second predetermined threshold are the same threshold.

8. The automated method of claim 2 wherein the first predetermined threshold and the second predetermined threshold are defined in relationship to a frequency content of a baseline of R-wave electrogram signals for the patient.

9. The automated method of claim 2 wherein the measure of the frequency content is a direct measure of frequency content of the electrogram signal.

10. The automated method of claim 2 wherein the measure of the frequency content is a relative measure based on one or more comparisons of the electrogram signals in the corresponding analysis window before and after a frequency-analysis step that modifies the electrogram signal based on frequency content.

11. The automated method of claim 1 wherein an electrogram signal in an analysis window is determined to be a high-frequency electrogram signal or a low-frequency electrogram signal in relationship to a ratio of a measure of the frequency content of said electrogram signal to the corresponding measure of frequency content of a baseline of R-wave electrogram signals for the patient.

12. The automated method of claim 11 wherein the measure of the frequency content is a differential analysis using a difference method or a derivative.

13. The automated method of claim 11 wherein the differential analysis comprises:
processing the electrogram signal in the analysis window using analog-to-digital conversion and bandpass filtering to generate an output electrogram signal;
applying a differential filter to the output electrogram signal to generate a set of outputs; and
using a peak detector to identify one or more maximum peaks of the set of outputs from the differential filter from which the ratio is determined.

14. The automated method of claim 3 in which, if a NPSI is determined to be a more specific NPSI, an additional analysis is performed, and if one or more specific criteria are met during the additional analysis, a lead system condition alert is generated.

15. The automated system of claim 14 in which the additional analysis comprises immediate performance of a measurement of lead impedance or impedance variability.

16. The automated system of claim 14 in which the additional analysis is performed of the electrogram signals in the two analysis windows corresponding to the sensed events that begin and end the NPSI; and
the additional analysis includes determining if the electrogram signal in either of the two analysis windows saturate a sense amplifier used for sensing the electrogram signal.

17. The automated method of claim 1 wherein the processor system is a processor within the cardiac implantable electrical device and the electrogram signal in the corresponding analysis windows is analyzed by the processor in real-time once the NPSI is determined.

18. The automated method of claim 1 wherein the processor system is a processor in a programmer or a remote monitoring network, and wherein a set of NPSIs is communicated from the cardiac implantable electrical device to the programmer or the remote monitoring network, and wherein the electrogram signal in the corresponding analysis windows of each of the set of NPSIs is then analyzed on a batch basis by the processor.
